(19) United States
(12) Patent Application Publication
(10) Pub. No.: US 2021/0196130 A1
(43) Pub. Date: Jul. 1, 2021

(54) METHOD FOR ESTIMATING THE PHYSICAL CONDITION OF A PERSON

(71) Applicant: Georg SCHMIDT, Munchen (DE)

(72) Inventor: Georg SCHMIDT, Munchen (DE)

(21) Appl. No.: 17/027,105

(22) Filed: Sep. 21, 2020

(30) Foreign Application Priority Data
Sep. 20, 2019 (DE) 102019006617.0
May 19, 2020 (DE) 102020002998.1

(51) Int. Cl. A61B 5/0205; A61B 5/1455

(52) U.S. Cl. CPC A61B 5/0205; A61B 5/14551

(57) ABSTRACT

The invention relates to a method for estimating the physical condition of a person by evaluating several parameters of the cardiovascular system by means of measurement signals in order to estimate the functionality of control of the cardiovascular system by the autonomous nervous system. Only one measurement signal, which contains information about the heart beat sequence and the force of contraction of the heart, is used to evaluate the parameters. The measurement signal is preferably the photoplethysmographic signal (PPG signal), which is used to detect the pulse rate and oxygen saturation of the tissue.

The invention relates to a method for estimating the physical condition of a person, in particular of the cardiovascular system.

Such methods are used to determine and measure functional parameters of the cardiovascular system. The condition of the person can be derived from the measurement signals.

For example, it is known from DE 10 2008 015 942 A1 that the measurement signals of an electrocardiogram (ECG) are used to evaluate a functional parameter that can be assigned to the autonomous function of the nervous system over time, in this case the heart rate and the associated deceleration capacity (DC).

In THE LANCET, Vol.353, Apr. 24, 1999, pages 1390 ff. the Heart Rate Turbulence (HRT) was described as a functional parameter with the help of 24-hour electrocardiograms (ECG) and evaluated for the condition of the respective person. HRT describes the physiological response of the heart rate to individual extrasystoles and is characterized in a healthy person by initial heart rate acceleration followed by deceleration. These significant phases are not or only slightly pronounced in a high-risk condition of the examined person.

Respiratory rate was also studied to determine a person's condition. For this purpose, the signals of a piezoelectric sensor arranged in a chest band were evaluated together with angiographic data, if applicable; see Petra Barthel et al. in European Heart Journal (2013) 34, pages 1644-1650.

Some time ago, the proposal was made to estimate the condition of a person according to the so-called polyscore. According to the polyscore, several functional parameters are measured and evaluated. These are, in particular, extrasystoles, ventricular and superventricular extrasystoles (ES), Heart Rate Turbulence (HRT), Deceleration Capacity (DC), Post-Extrasystolic Potentiation (PESP), Baroreflex Sensitivity (BRS), the Respiratory Rate (AF) and, in the process, sinus arrhythmia during exhalation (Exhalation Triggered Arrhythmia ETA) or during the entire respiratory phase (Respiratory Sinus Arrhythmia RSA).

Each of these functional parameters can be measured and evaluated with known methods, often in a non-invasive manner. In addition to those mentioned above, a large number of publications are available on this subject. Investigations are always necessary to obtain different measurement signals with different devices, which usually record ECG, blood pressure and often also respiration. A complete and accurate examination of the condition of a person, in which several functional parameters of the cardiovascular system would have to be measured and evaluated, requires a great deal of equipment and time.

The invention is based on the task of simplifying the procedure for estimating the physical condition of a person while achieving a high degree of accuracy in the estimation.

This is achieved by the features of claim 1.

According to the invention, several functional parameters are evaluated from a single measurement signal in order to estimate the functionality and quality of the control of the cardiovascular system. For evaluation of the parameters, the measurement signal contains information about the heart beat sequence and the force of contraction of the heart.

The single measurement signal is preferably the quasiperiodic photoplethysmographic signal (PPG signal), which, often used, is used to record the pulse and thus the heart rate and the oxygen saturation of the tissue. The signal is obtained with a simple device, e.g., a so-called wearable, a finger clip, a fitness watch, an ear sensor etc.

It has been found that if this single measurement signal is cleverly evaluated, many functional parameters that are important for estimating the condition of a person and all the above-mentioned parameters of the polyscore can be recorded. Thus, the effort for the procedure according to the invention is significantly lower than before, without compromising the accuracy compared to previous complex methods.

The PPG signal and its amplitude correlate very well with the blood pressure signal on short time scales. The time intervals between the individual beats correspond to the heart rate and can be evaluated like the intervals between the RR peaks in an ECG. The amplitude modulation can be interpreted as fluctuation of the blood pressure. Furthermore, the breathing signal can be derived from the envelope of the amplitudes of the PPG signal, which has to be generated by calculation, with the breathing rate.

Thus, short-term blood pressure fluctuations and also extrasystoles, whether ventricular or supra-ventricular, can be detected from the PPG signal, since the signal “pauses” at these points between two regular beats or has a short and a longer reduced beat. This use of the PPG signal is proposed in DE 10 2018 022 282.0, which was not previously published.

According to the invention, the amplitudes, the time intervals between the individual signals or amplitudes or a combination of both quantities as well as their variations are evaluated stroke by stroke for the PPG signals, depending on the functional parameters to be determined. This can be done in accordance with known methods.

With the evaluation of the time intervals, the functional parameters arrhythmia, deceleration capacity and heart rate turbulence are determined. With the evaluation of the parameters baroreflex sensitivity, respiratory rate, arrhythmia triggered on exhalation and post-extrasystolic blood pressure potentiation, the time intervals and amplitudes are taken into account.

It is well known that there is a relationship, a robust correlation, between these variables, time interval and subsequent amplitude of the PPG signal; see D. Sinnecker et al, International Journal of Cardiology 182 (2015), 315-320. Artifacts, i.e., technical disturbances in the signal do not show this correlation. Signal areas contaminated with artifacts can therefore be reliably identified on the basis of the lack of correlation and excluded from an analysis.

Known averaging procedures can be used in the evaluation, in which significant values of the amplitudes, time intervals or a combination of these quantities of the PPG signal are used as so-called anchors for the evaluation of the condition of a person for the respective function parameter and signal parts around these anchors are averaged by superposition.

In the averaging procedure, for example, segments are formed from individual beats of the PPG signal around the anchor before and/or after it. Several segments are then superimposed and evaluated with the averaging procedure. The averaging procedure is, for example, the well-known PRSA method, where PRSA stands for “phase-rectified signal averaging.” In addition to the univariate PRSA method for evaluating one variable, the bivariate BPRSA method is also used for evaluating several variables.

### BRIEF DESCRIPTION

The invention is explained in more detail on the basis of the drawing. In the drawing:

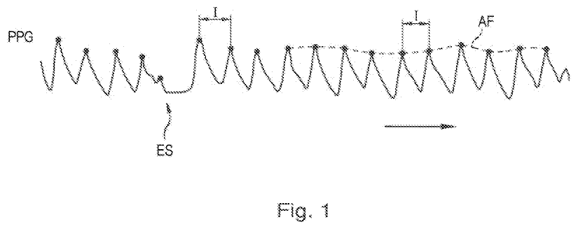

FIG. 1 depicts, schematically, a PPG signal, in which individual characteristics considered in the invention are drawn in, as well as a part of the breath signal AF; and

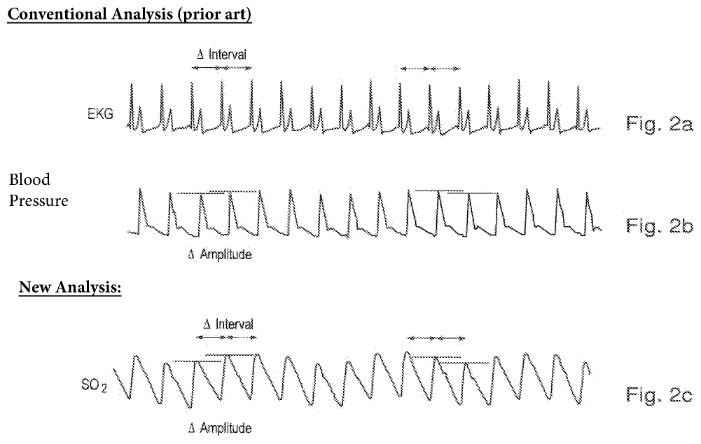

FIG. 2 with lines a), b) and c), where lines a) and b) show the ECG signal and the blood pressure signal, respectively, which are required for a conventional analysis of the condition of a person, and line c) shows the PPG signal which is used for the analysis according to the invention.

### DETAILED DESCRIPTION

FIG. 1 shows a part of a PPG signal curve over time, with an extrasystole ES and indicated intervals I between two signals.

The interval I is determined either as the time interval between two maxima or minima or by the distance between two points with the maximum slope, i.e., the maximum of the first derivative.

According to FIGS. 2a and 2b, at least ECG and blood pressure signals are used for a conventional analysis of the condition of a person.

According to FIG. 2c, only the PPG signal, which is designated SO2—for oxygen saturation—, is required for an evaluation according to the invention. Decisive for the evaluation are the differences of the time intervals “delta interval” between the signal beats and the amplitude values “delta amplitude.”

The following applies to the evaluation of the individual function parameters:

Heart Rate Turbulence (HRT) is the physiological response of the heart rate to individual ventricular extrasystoles ES; it is a sign of arrhythmia. In a healthy person, the heart rate first becomes faster for a few intervals, after which deceleration occurs. By plotting the intervals I determined by the PPG signal beat by beat after an extrasystole and the resulting heart rate, a few, e.g., five consecutive points within the scatter plotted for 15 intervals, for example, show the slope of the curve, which is an indication of the person's condition: the lower the slope, the more risky the person's condition; see the above-mentioned article in THE LANCET.

To take into account the Deceleration Capacity DC, for each interval I of the PPG signal it is determined whether it is longer than the previous one. Of course, a certain threshold can be specified here, which must exceed the longer interval. The longer interval is called the anchor. Around each anchor some, e.g., five, intervals are recorded in positive and negative direction. The signal sequences are called segments. All segments thus determined are superimposed in an averaging process with the anchors as center. Preferably, a phase-averaged univariate PRSA signal is formed for the variable “interval” (phase-rectified signal averaging), which makes a more or less large visible jump around the anchor center depending on the condition of the person. The PRSA method is described for the deceleration capacity determined by conventional means, e.g., by Axel Bauer et al. in the journal Lancet, Vol.367 dated May 20, 2006.

To take into account the post-extrasystolic blood pressure potentiation PESP, it is determined, after an extrasystole ES, for the first following amplitude whether it exceeds the amplitudes of the following (or previous) e.g., ten averaged signal amplitudes. In a subsequent evaluation, e.g., a PRSA evaluation, this maximum of the first signal becomes the anchor. The higher the value of the first signal that exceeds the mean value, the less effectively can the person's condition be evaluated; for a conventional evaluation see D. Sinnecker et al., Journal of the American Heart Association 2014;:e000857, Jun. 3, 2014.

To investigate Baroreflex Sensitivity (BRS), the intervals that exceed a mean value of the amplitude of the PPG signal are determined as anchors. This interval in turn becomes an anchor for PRSA averaging. Since the PPG signal correlates well with blood pressure and this depends on respiration, a good evaluation is possible.

The quasiperiodic respiratory signal AF is represented by the envelope of the maxima of the PPG signal and, accordingly, is generated by calculation and evaluated in frequency. The respiratory signal AF is partly indicated by dashed lines in FIG. 1. The generated respiratory signal is used to derive the function parameter ETA, i.e., the arrhythmia triggered by exhalation. In the time period following the maximum of the breath signal, which is assigned to the exhalation phase, it is examined whether a new PPG signal begins. This signal in turn becomes an anchor for the averaging process, e.g., the described evaluation according to the PRSA method.

With an estimation according to the invention, which is possible only from the PPG signal, the condition and prognosis for a person's future condition can be determined. This risk assessment then makes it possible to reliably decide whether further diagnostics are indicated or whether the person needs treatment.

The measurement signal can also be another modulating signal if it provides similar information, e.g., an actimetric signal obtained with an accelerometer.

1. Method for estimating the physical condition of a person, the method comprising:
evaluating several parameters of a cardiovascular system of the person by means of a measurement signal, wherein the measurement signal contains information about a heartbeat sequence and a force of contraction of a heart of the cardiovascular system; and
estimating, based on evaluating, a functionality of control of the cardiovascular system through an autonomous nervous system.

2. The method of claim 1, wherein the measurement signal is a photoplethysmographic signal used to detect the heartbeat and oxygen saturation of tissue.

3. The method of claim 1, further comprising:
selecting significant points for a quasiperiodic measurement signal;
determining, based on a time interval between corresponding points of successive signals, a time interval, wherein at least one of a maximum, a minimum and a point with a maximum rate of change, are selected as significant points.

4. The method of claim 1, wherein at least one of time intervals and signal values of amplitudes of the measurement signal are used to evaluate the parameters.

5. The method of claim 4, wherein the intervals that differ significantly from other intervals serve as an anchor for a segment of at least one or more intervals at least one of before and after the anchor, such that several segments are superimposed and averaged to form a parameter via an averaging method, the averaging method serving a phase-rectified signal averaging method.

6. The method of claim 5, wherein a parameter for Heart Rate Turbulence is determined by plotting a sequence of intervals occurring after an extrasystole and determining a slope of the straight lines connecting said intervals.

7. The method of claim 5, wherein for investigation of a deceleration capacity, the intervals are determined in each case which are longer than a preceding interval, said intervals are applied as an anchor together with adjacent intervals as segments and are applied around an anchor for averaging with further segments.

8. The method of claim 5, wherein for examination of post-extrasystolic blood pressure potentiation after an extrasystole for a subsequent or preceding segment of the measurement signals, it is determined whether a first amplitude exceeds a certain number of at least one of subsequent and preceding amplitudes and whether a course of a connecting line of amplitude peaks is wavy or smooth in a good condition of the person.

9. The method of claim 5, wherein for investigation of baroreflex sensitivity, the intervals are selected as anchors for the averaging, said intervals exceeding a mean value of the amplitude of the measurement signal of a considered signal sequence.

10. The method of claim 5, wherein for investigation of a respiratory signal, an envelope of maxima corresponding to a respiratory signal is generated and a frequency of said signal is determined for a condition of the person.

11. The method of claim 10, wherein, for investigation of exhalation-triggered arrhythmia, an interval of the photoplethysmographic signal is selected as an anchor for averaging, starting in an exhalation phase after a maximum of the respiratory signal.
